(19) United States
(12) Patent Application Publication
(10) Pub. No.: US 2020/0129511 A1
(43) Pub. Date: Apr. 30, 2020

(54) COMPOUNDS AND METHODS FOR TREATMENT OF NAFLD AND NASH

(71) Applicant: Arena Pharmaceuticals, Inc., San Diego, CA (US)

(72) Inventors: John W. Adams, San Diego, CA (US); Que Liu, San Diego, CA (US)

(21) Appl. No.: 16/624,198

(22) PCT Filed: Jun. 19, 2018
(86) PCT No.: PCT/US2018/038319

§ 371 Date: Dec. 18, 2019

Related U.S. Application Data
(60) Provisional application No. 62/522,045, filed on Jun. 19, 2017.

(51) Int. Cl. A61K 31/506; A61K 9/20; A61K 9/48; A61P 1/16

(52) U.S. Cl. CPC A61K 31/506; A61P 1/16; A61K 9/48; A61K 9/20

(57) ABSTRACT

The present invention relates to 4-[6-(6-Methanesulfonyl-2-methyl-pyridin-3-ylamino)-5-methoxy-pyrimidin-4-yloxy]-piperidine-1-carboxylic acid isopropyl ester (Compound 1), pharmaceutically acceptable salts, solvates, and hydrates thereof that modulate the activity of the GPR119 receptor. Compound 1 and pharmaceutical compositions thereof are directed to methods useful in the treatment of non-alcoholic steatohepatitis (NASH), non-alcoholic fatty liver disease (NAFLD), and conditions related thereto.

### FIELD OF THE INVENTION

The present invention relates to 4-[6-(6-Methanesulfonyl-2-methyl-pyridin-3-ylamino)-5-methoxy-pyrimidin-4-yloxy]-piperidine-1-carboxylic acid isopropyl ester (Compound 1), pharmaceutically acceptable salts, solvates, and hydrates thereof that modulate the activity of the GPR119 receptor. Compound 1 and pharmaceutical compositions thereof are directed to methods useful in the treatment of non-alcoholic fatty liver disease (NAFLD), non-alcoholic steatohepatitis (NASH), and conditions related thereto.

### BACKGROUND OF THE INVENTION

GPR119 (e.g., human GPR119, GENBANK® Accession No. AAP72125 and alleles thereof; e.g., mouse GPR119, GENBANK® Accession No. AY288423 and alleles thereof) is a GPCR located at chromosome position Xp26.1 (Fredricksson, R. et al., “Seven evolutionarily conserved human rhodopsin G protein-coupled receptors lacking close relatives”, FEBS Lett., 554:381-388 (2003)) and is selectively expressed on pancreatic beta cells. The receptor is coupled to Gs, and when stimulated, produces an elevation in cAMP in a variety of cell types including β-cell-derived insulinomas (Soga, T. et al., “Lysophosphatidylcholine enhances glucose-dependent insulin secretion via an orphan G-protein-coupled receptor”, Biochem. Biophys. Res. Comm., 326:744-751 (2005), PCT Publication Nos. WO 04/065380, WO 04/076413, WO 05/007647, WO 05/007658, WO 05/121121, and WO 06/083491). The receptor has been shown to be localized to the β-cells of the pancreas in a number of species as well as in specific cell types of the gastrointestinal tract. Activation of GPR119, with agonist ligands such as lysophosphatidylcholine, produce a glucose dependent increase in insulin secretion from primary mouse islets and various insulinoma cell lines such as NIT-1 and HIT-T15 (Soga, T. et al., “Lysophosphatidylcholine enhances glucose-dependent insulin secretion via an orphan G-protein-coupled receptor”, Biochem. Biophys. Res. Comm., 326:744-751 (2005); Chu, Z. L. et al., “A role for β-cell-expressed G protein-coupled receptor 119 in glycemic control by enhancing glucose-dependent insulin release”, Endocrinology, 148 (6):2601-2609 (2007)). In the literature, GPR119 has also been referred to as RUP3 (see, International Application WO 00/31258) and as Glucose-Dependent Insulinotropic Receptor GDIR (see, Jones, et al. Expert Opin. Ther. Patents (2009), 19(10): 1339-1359). GPR119 agonists also stimulate the release of Glucose-dependent Insulinotropic Polypeptide (GIP), Glucagon-Like Peptide-1 (GLP-1), and at least one other L-cell peptide, Peptide YY (PYY) (Jones, et al. Expert Opin. Ther. Patents (2009), 19(10): 1339-1359); for specific references related to GPR119 agonists and the release of: GIP, see Shah, Current Opinion in Drug Discovery & Development, (2009) 12:519-532; Jones, et al., Ann. Rep. Med. Chem., (2009) 44:149-170; WO 2007/120689; and WO 2007/120702; GLP-1, see Shah, Current Opinion in Drug Discovery & Development, (2009) 12:519-532; Jones, et al., Ann. Rep. Med. Chem., (2009) 44:149-170; Schwartz et al., Cell Metabolism, 2010, 11:445-447; and WO 2006/076231; and PYY, see Schwartz et al., Cell Metabolism, 2010, 11:445-447; and WO 2009/126245.

### NAFLD and NASH

Non-alcoholic fatty liver disease (NAFLD) is a disorder affecting as many as 1 in 3-5 adults and 1 in 10 children in the United States, and refers to conditions where there is an accumulation of excess fat in the liver of people who drink little or no alcohol. The most common form of NAFLD is a non-serious condition called hepatic steatosis (fatty liver), in which fat accumulates in the liver cells: although this is not normal, by itself it probably does not damage the liver. Fatty liver disease is generally detected by observation of elevated serum concentrations of liver-specific enzymes such as alanine aminotransferase (ALT) and aspartate aminotransferase (AST), which serve as indices of hepatocyte injury, as well as by presentation of symptoms which include fatigue and pain in the region of the liver, though definitive diagnosis often requires a biopsy.

NAFLD most often presents itself in individuals with a constellation of risk factors related to metabolic syndrome, which is characterized by elevated fasting plasma glucose (FPG) with or without intolerance to post-prandial glucose, being overweight or obese, high blood lipids such as cholesterol and triglycerides (TGs) and low high-density lipoprotein cholesterol (HDL-C) levels, and high blood pressure; but not all patients have all the manifestations related to metabolic syndrome. Obesity is thought to be the most common cause of NAFLD; and some experts estimate that about two-thirds of obese adults and one-half of obese children may have fatty liver. Many individuals with NAFLD have no symptoms and a normal physical examination (although the liver may be slightly enlarged); children may exhibit symptoms such as abdominal pain and fatigue, and may show patchy dark skin discoloration (acanthosis nigricans). The diagnosis of NAFLD is usually first suspected in an overweight or obese person who is found to have mild elevations in their liver blood tests during routine testing, though NAFLD can be present with normal liver blood tests, or incidentally detected on imaging investigations such as abdominal ultrasound or CT scan. It is confirmed by imaging studies, most commonly a liver ultrasound or magnetic resonance imaging (MRI), and exclusion of other causes.

Some people with NAFLD may develop a more serious condition called non-alcoholic steatohepatitis (NASH): about 2-5% of adult Americans and up to 20% of those who are obese may suffer from NASH. In NASH, fat accumulation in the liver is associated with inflammation and different degrees of scarring. NASH is a potentially serious condition that carries a substantial risk of progression to end-stage liver disease, cirrhosis and hepatocellular carcinoma. Some patients who develop cirrhosis are at risk of liver failure and may eventually require a liver transplant.

One of skill in the art will recognize established scoring systems for NAFLD and NASH. For example, NAFLD may be differentiated from NASH by a NAFLD Activity Score (NAS) or a Steatosis, Activity, and Fibrosis (SAF) score. In some embodiments, the sum of the histopathology scores of a liver biopsy for steatosis (e.g., 0 to 3), lobular inflammation (e.g., 0 to 2), and hepatocellular ballooning (e.g., 0 to 2). In some embodiments, steatosis is 0, steatosis is 1, steatosis is 2, steatosis is 3, ballooning degeneration is 0, ballooning degeneration is 1, ballooning degeneration is 2, lobular inflammation is 0, lobular inflammation is 1, lobular inflammation is 2, lobular inflammation is 3, fibrosis is 0, fibrosis is 1, fibrosis is 2, fibrosis is 3, fibrosis is 4, or a combination thereof. In some embodiments, the scoring system includes steatosis, ballooning degeneration, lobular inflammation, and any combination of the numerical score for each as described above. In some embodiments, the scoring system includes steatosis, ballooning degeneration, lobular inflammation, fibrosis, and any combination of a numerical score for each as described above. In some embodiments, a NAS of <3 corresponds to NAFLD, 3-4 corresponds to borderline NASH, and ≥5 corresponds to NASH. The biopsy can also be scored for fibrosis (e.g., 0 to 4).

NASH is a leading cause of end-stage liver disease; while NAFLD, and to an even greater degree NASH, are intimately related to metabolic syndrome, including insulin resistance (pre-diabetes) and type 2 diabetes mellitus (T2DM), and abdominal obesity. T2DM has been the most prominent predictor for a poor prognosis in NAFLD, whereas elevated liver enzymes are considered unreliable. NASH develops much more frequently in the presence of longstanding T2DM, and most patients with cryptogenic cirrhosis are obese and/or diabetic. Studies have demonstrated that 60% of patients with T2DM and NAFLD had biopsy-proven NASH, and that advanced hepatic fibrosis was present in 75% of those with diabetes and hypertension compared to only 7% without either condition. Haukeland reported that impaired glucose tolerance (IGT) and T2DM were the only independent risk factors for severe NAFLD and NASH, increasing the odds ratio almost 4-fold (“Abnormal glucose tolerance is a predictor of nonalcoholic steatohepatitis and fibrosis in patients with non-alcoholic fatty liver disease”, Scand. J. Gastroenterol., 40, 1469-1477 (2005)). Mofrad reported a study that demonstrated the lack of predictive value for elevated liver transaminases to diagnose NASH in patients with NAFLD and found T2DM to be the only factor independently associated with an increased risk of advanced fibrosis (“Clinical and histological spectrum of nonalcoholic fatty liver disease associated with normal ALT levels”, Hepatology, 37, 1286-1292 (2003)). Thus, NASH is an overlooked complication of T2DM that is frequently associated with fibrosis and in approximately 10% of patients results in cirrhosis; while the risk of hepatocellular carcinoma is also increased in patients with T2DM and NASH. Patients with NAFLD and NASH usually demonstrate mixed dyslipidemia and the other metabolic derangements described above, including an atherogenic low-density lipoprotein (LDL) phenotype consisting of predominantly of small dense particles. Both metabolic syndrome and NAFLD/NASH are characterized by increased cardiovascular inflammation as measured by elevations in high sensitivity C-reactive protein (hsCRP) and other inflammatory cytokines.

There is significant worldwide incidence of obesity, metabolic syndrome, pre-diabetes and diabetes, with the prevalence of diabetes worldwide predicted to double to 366 million by 2030. The US population with diabetes has been estimated to be 37.7 million (14.5%) by 2031. Because approximately 70% of persons with T2DM have a fatty liver, and the disease follows a more aggressive course with necroinflammation and fibrosis (i.e., NASH) in diabetes, the epidemiology of diabetes suggests significant increases in NASH and chronic liver disease. Using MRI for the noninvasive assessment of hepatic steatosis, the prevalence of NAFLD, when defined as liver fat >5%, has been estimated to be 34% in the USA or approximately 80 million people, and as many as two out of three obese subjects. However, this prevalence is believed to be much higher in T2DM.

Recently it was reported that oral administration of MBX-2982 (a GPR119 agonist) in mice fed a high-fat diet potently inhibited hepatic lipid accumulation and expression levels of sterol regulatory element binding protein (SREBP-1) and lipogenesis-related genes, whereas the hepatic antilipogenesis effects of MBX-2982 were abolished in GPR119 KO mice (Yang et al. “GPR119: a promising target for nonalcoholic fatty liver disease”, FASEB J. 30, 324-335 (2016)), suggesting that the MBX-2982 alleviated hepatic steatosis or lessened the abnormal retention of lipids in the liver by inhibiting SREBP-1-mediated lipogenesis in hepatocytes.

### Compound 1: 4-[6-(6-Methanesulfonyl-2-methyl-pyridin-3-ylamino)-5-methoxy-pyrimidin-4-yloxy]-piperidine-1-carboxylic acid isopropyl ester

As described herein, the compound 4-[6-(6-Methanesulfonyl-2-methyl-pyridin-3-ylamino)-5-methoxy-pyrimidin-4-yloxy]-piperidine-1-carboxylic acid isopropyl ester (Compound 1) is a potent (EC₅₀ of 46 nM, HTRF cAMP assay, (human)) and selective (no significant activity in a standard CEREP receptor selectivity panel when tested at 1 μM), orally bioavailable investigational drug candidate of the GPR119 receptor for the treatment of non-alcoholic fatty liver disease (NAFLD), non-alcoholic steatohepatitis (NASH), and conditions related thereto.

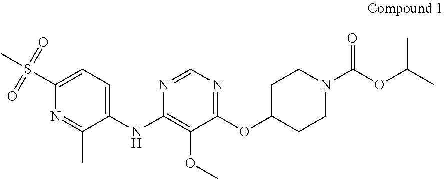 [SMILES: COc1c(Nc2ccc(S(C)(=O)=O)nc2C)ncnc1OC1CCN(C(=O)OC(C)C)CC1]

In preclinical studies, Compound 1 showed significant reduction of AUC_{glu} (FIG. 1) in normal lean mice during an oral glucose tolerance test (oGTT) at 1 mg/kg and 10 mg/kg p.o. In addition, in the Sprague-Dawley rat, treatment with Compound 1 (3-30 mg/kg PO) significantly improved glucose handling and markedly reduced the AUC_{glu} during oGTT (see FIG. 2). See Example 2 for a representative procedure used for the oGTT. More extensive in vivo studies showed Compound 1 to have highly favorable absorption, distribution, metabolism, and excretion (ADME) characteristics. For example, exposure was shown to be high after oral dosing particularly in the mouse, consistent with the excellent effect observed in the oGTT in that species. Additional ADME data are shown in the table below.

| Species | Dose | tₘₐₓ | Cₘₐₓ | AUC_{inf} | t_{1/2} | |
|---|---|---|---|---|---|---|
| (Dose) | Vehicle | (h) | (μg/mL) | (h μg/mL) | (h) | % F |
| Male C57B1 mouse | 40% | 2 | 25.833 | 319.953 | 5.9 | — |
| (10 mg/kg) | HPβCD | | | | | |
| Male Sprague- | 40% | 1 | 1.39 | 12.471 | 4.3 | 72 |
| Dawley rat | HPβCD | | | | | |
| (10 mg/kg) | | | | | | |
| Male beagle dog | 100% | 8 | 1.983 | 43.743 | 10.9 | 22.1 |
| (7.85 mg/kg) | PEG400 | | | | | |
| Male cynomolgus | 100% | 6 | 4.803 | 64.18 | 4.4 | 68.8 |
| monkey (10 mg/kg) | PEG400 | | | | | |

Further studies confirmed the activity of Compound 1 in several well established diabetic rodent models as well as in normal monkey in the same dose range; see the table below.

| | Blood glucose | Compd exposure | |
|---|---|---|---|
| | AUC (% of | upon oGTT | P value of |
| Animal model (Dose) | vehicle group) | (ng/mL) | t-test |
| ob/ob Mice (3 mg/kg) | 78.4 ± 5.1 | Not tested | P < 0.01 |
| SD rats (3 mg/kg) | 85.1 ± 1.9 | 482 ± 116 | P < 0.01 |
| ZDF rat (3 mg/kg) | 72.8 ± 6.3 | 1592 ± 128 | P < 0.01 |
| Cynomolgus monkey | 82.8 ± 1.7 | 112 ± 16.9 | P < 0.001 |
| (3 mg/kg) | | | |

Furthermore, Compound 1 was observed to have high permeability across Caco-2 monolayers (A to B: 26×10⁻⁶ cm/s and B to A: 15×10⁻⁶ cm/s) and was stable in rat and human liver microsomes (t_{1/2}>60 min). As stated above, Compound 1 also showed no significant activity in a standard CEREP receptor selectivity panel when tested at 1 μM. In addition, Compound 1 showed no significant inhibition of hERG channel binding (IC₅₀>10 μM) and in a patch clamp study Compound 1 showed an IC₅₀ of 13±0.2 μM. In the anesthetized Guinea Pig, treatment with Compound 1 did not produce any dose-related, statistically significant effects on mean arterial blood pressure (MAP), heart rate (HR) or on the electrocardiogram (ECG) at cumulative doses up to 5 mg/kg i.v., when compared to vehicle controls. Preliminary safety studies in rat (14-day) and dog (7-day) revealed no obvious liabilities.

Compound 1 was selected for clinical evaluation. Randomized, double-blind, placebo-controlled Phase 1 clinical trials were conducted to evaluate the safety, tolerability, pharmacodynamics, and pharmacokinetics of Compound 1 (2.5-800 mg) in healthy male volunteers. The systemic exposure of Compound 1 in plasma increased in proportion to the dose and was not influenced by coadministration of food. The terminal elimination half-life was ˜13 h when administered as an oral suspension formulation. Compound 1 was determined to be well tolerated and was not associated with hypoglycemia. As compared with placebo, an oral single-dose of Compound 1 increased postmeal plasma glucagon-like peptide 1 (GLP-1), glucose-dependent insulinotropic peptide (GIP), and peptide YY (PYY) concentrations but did not significantly decrease glucose excursion or increase insulin secretion. However, in a graded glucose infusion study, Compound 1 was shown to induce a higher insulin secretion rate (ISR) relative to placebo at elevated plasma glucose levels. These studies provide evidence for the potential efficacy of Compound 1 (also referred to as JNJ-38431055) as an anti-diabetes agent in humans; see, Katz et al., “Effects of JNJ-38431055, a novel GPR119 receptor agonist, in randomized, double-blind, placebo-controlled studies in subjects with type 2 diabetes” Clin. Pharm. Ther., 90(4), 685-692 (2011).

A subsequent clinical study was conducted in T2DM patients. This was a randomized, double-blind, placebo- and positive-controlled, single-dose cross-over study and a randomized, double-blind, placebo-controlled multiple-dose parallel design study. Two different studies were performed involving 25 and 32 different male and female subjects, with ages of 25-60 years, mean body mass index between 22 and 39.9 kg/m² who had T2DM diagnosed 6 months to 10 years before screening. Compound 1, either 100 or 500 mg, or sitagliptin (100 mg) as a single-dose or Compound 1 (500 mg) once daily for 14 consecutive days were tested. Compound 1 was well tolerated and not associated with hypoglycemia. Plasma systemic exposure of Compound 1 increased with increased dose and was approximately two-fold greater after multiple-dose administration and attained steady-state after approximately 8 days. Compared with placebo, single-dose administration of oral Compound 1 decreased glucose excursion during an oral glucose tolerance test. Multiple dosing of Compound 1 increased post-meal total glucagon-like peptide 1 and gastric insulinotropic peptide concentrations compared to baseline; see, Katz et al., “Effects of JNJ-38431055, a novel GPR119 receptor agonist, in randomized, double-blind, placebo-controlled studies in subjects with type 2 diabetes”, Diabetes, Obesity and Metabolism 14: 709-716, (2012).

There are currently no drugs approved to prevent or treat NAFLD or NASH. Although a number of pharmacological interventions have been studied for the treatment of NAFLD or NASH with limited overall benefit, there still remains a significant unmet clinical need for an effective and well-tolerated treatment that can prevent or slow the progression of NAFLD and NASH.

Citation of any reference throughout this application is not to be construed as an admission that such reference is prior art to the present application.

### SUMMARY OF THE INVENTION

One aspect of the present invention is directed to, inter alia, methods of treating non-alcoholic fatty liver disease (NAFLD) or a condition related thereto in an individual in need thereof comprising administering a therapeutically effective amount of 4-[6-(6-Methanesulfonyl-2-methyl-pyridin-3-ylamino)-5-methoxy-pyrimidin-4-yloxy]-piperidine-1-carboxylic acid isopropyl ester (Compound 1), or a pharmaceutically acceptable salt, hydrate, or solvate thereof.

In some embodiments, the NAFLD is simple steatosis (NAFL). In some embodiments, the NAFLD is steatohepatitis (NASH). In some embodiments, the NAFLD is liver cirrhosis. In some embodiments, the NAFLD is NASH with a degree of fibrosis selected from F1, F2, F3, and F4 fibrosis. In some embodiments, the NAFLD is NASH with F4 fibrosis.

In some embodiments, the individual has at least one condition selected from hepatic steatosis, lobular inflammation, and hepatocellular ballooning.

In some embodiments, the NAFLD is characterized by a NAFLD activity score (NAS) greater than or equal to 4.

In some embodiments, treating NAFLD is decreasing the NAS at least 1, 2, or 3 points. In some embodiments, treating NAFLD is reducing the worsening of fibrosis. In some embodiments, treating NAFLD is reversing steatohepatitis. In some embodiments, treating NAFLD is ceasing progression to F3 or F4 fibrosis. In some embodiments, treating NAFLD is resolving NASH. In some embodiments, treating NAFLD is not worsening liver fibrosis. In some embodiments, treating NAFLD is reducing the risk of liver-related death. In some embodiments, treating NAFLD is improving liver fibrosis by at least one stage. In some embodiments, treating NAFLD is improving cardiometabolic and/or liver markers.

In some embodiments, the individual has been determined to have NALFD using a NAFLD fibrosis score.

### BRIEF DESCRIPTION OF THE DRAWINGS

FIG. 1 shows the effect of Compound 1 (1 mg/kg and 10 mg/kg) on glucose excursion in an oGTT in male C57bl/6j mice. Left panel: Blood glucose levels at various times relative to glucose injection. Middle panel: AUC's derived from Left panel. Right panel: Percent change in area under the curve (AUC). For statistical analysis, a one-way ANOVA was performed on AUC's. ANOVA revealed a significant main effect of Compound 1.

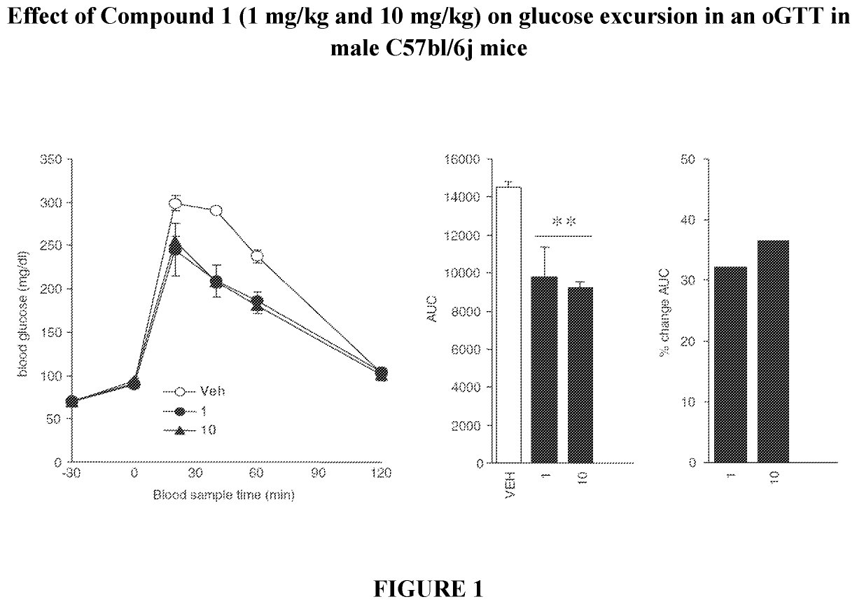

FIG. 2 shows the effect of Compound 1 (3-30 mg/kg p.o.) on glucose excursion in Sprague-Dawley rat after administration of an oral (p.o.) dose of glucose. Left panel, blood glucose levels at various times relative to glucose injection. Right panel, AUC's derived from the left panel (AUC, based on incremental changes post-bolus). For statistical analysis, a one-way ANOVA was performed on AUC's. ANOVA revealed a significant main effect of Compound 1.

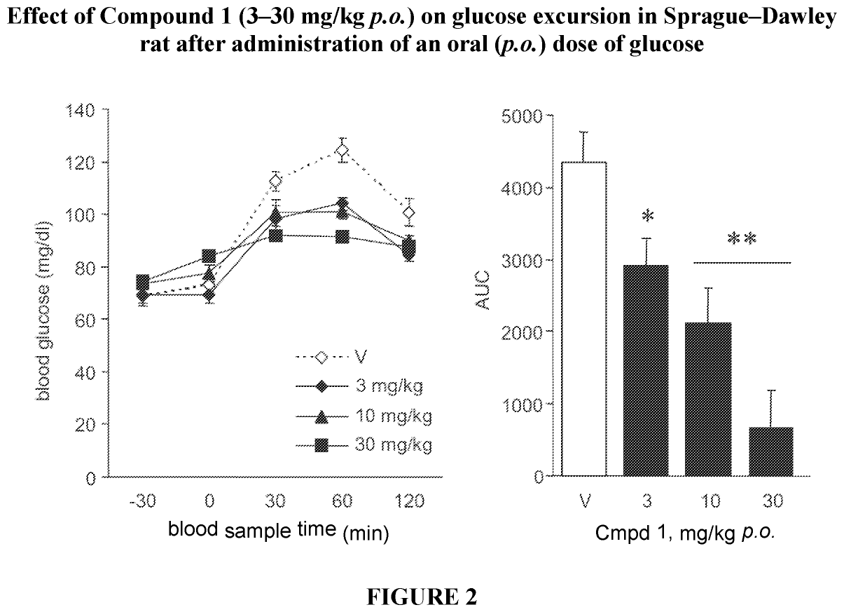

FIG. 3 shows a representative powder X-ray diffraction (PXRD) pattern measured as described herein of crystalline Form A-I for Compound 1.

FIG. 4 shows a representative powder X-ray diffraction (PXRD) pattern measured as described herein of crystalline Form A-IV for Compound 1.

FIG. 5 shows a representative powder X-ray diffraction (PXRD) pattern measured as described herein of crystalline Form A-VI for Compound 1.

FIG. 6 shows serum alanine aminotransferase (ALT) after treatment with Compound 1 and MBX (MBX-2982) in the High Fat Diet (HFD) Mouse Model.

### DETAILED DESCRIPTION OF THE INVENTION

In its various embodiments, the present invention is directed to, inter alia, methods of treating non-alcoholic fatty liver disease (NAFLD) or a condition related thereto in an individual in need thereof comprising administering a therapeutically effective amount of 4-[6-(6-Methanesulfonyl-2-methyl-pyridin-3-ylamino)-5-methoxy-pyrimidin-4-yloxy]-piperidine-1-carboxylic acid isopropyl ester (Compound 1), or a pharmaceutically acceptable salt, hydrate, or solvate thereof. In some embodiments, the individual is determined to have NAFLD by liver biopsy or by NAFLD fibrosis score. In some embodiments, the individual is determined to have NAFLD by liver biopsy. In some embodiments, the individual is determined to have NAFLD by NAFLD fibrosis score.

One aspect of the present invention relates to methods of treating non-alcoholic fatty liver disease (NAFLD) or a condition related thereto in an individual in need thereof comprising administering a therapeutically effective amount of 4-[6-(6-Methanesulfonyl-2-methyl-pyridin-3-ylamino)-5-methoxy-pyrimidin-4-yloxy]-piperidine-1-carboxylic acid isopropyl ester (Compound 1), or a pharmaceutically acceptable salt, hydrate, or solvate thereof.

Compound 1 and pharmaceutically acceptable salts, hydrates, and solvates thereof disclosed herein are useful in the treatment non-alcoholic fatty liver disease (NAFLD) or a condition related thereto. One skilled in the art will recognize that when a disorder, or a method of treatment, is disclosed herein, such disclosure encompasses second medical uses (e.g., Compound 1 and pharmaceutically acceptable salts, hydrates, and solvates thereof for use in the treatment of NAFLD or a condition related thereto; use of Compound 1 and pharmaceutically acceptable salts, hydrates, and solvates thereof for the treatment of NAFLD or a condition related thereto; and use of Compound 1 and pharmaceutically acceptable salts, hydrates, and solvates thereof in the manufacture of a medicament for the treatment of the disorder).

One aspect of the present invention relates to a compound selected from the following compound and pharmaceutically acceptable salts, solvates, and hydrates thereof:

4-[6-(6-Methanesulfonyl-2-methyl-pyridin-3-ylamino)-5-methoxy-pyrimidin-4-yloxy]-piperidine-1-carboxylic acid isopropyl ester (Compound 1),

for use in a method of treating non-alcoholic fatty liver disease (NAFLD) or a condition related thereto in an individual comprising administering a therapeutically effective amount of Compound 1, or a pharmaceutically acceptable salt, hydrate, or solvate thereof.

One aspect of the present invention relates to uses of a compound selected from the following compound and pharmaceutically acceptable salts, solvates, and hydrates thereof:

4-[6-(6-Methanesulfonyl-2-methyl-pyridin-3-ylamino)-5-methoxy-pyrimidin-4-yloxy]-piperidine-1-carboxylic acid isopropyl ester (Compound 1),

in the manufacture of a medicament for the treatment of non-alcoholic fatty liver disease (NAFLD) or a condition related thereto in an individual comprising administering a therapeutically effective amount of Compound 1, or a pharmaceutically acceptable salt, hydrate, or solvate thereof.

In some embodiments, the total daily dose of Compound 1 or a pharmaceutically acceptable salt thereof is about 2.5 mg to 800 mg.

In some embodiments, the daily therapeutically effective amount of Compound 1 or the pharmaceutically acceptable salt thereof is about 2.5 mg to 800 mg.

In some embodiments, the total daily dose of Compound 1 or a pharmaceutically acceptable salt thereof is about 100 mg to 800 mg.

In some embodiments, the daily therapeutically effective amount of Compound 1 or the pharmaceutically acceptable salt thereof is about 100 mg to 800 mg.

In some embodiments, the Compound 1 or a pharmaceutically acceptable salt thereof is administered at a frequency of 4 or less times per day.

In some embodiments, the Compound 1 or the pharmaceutically acceptable salt thereof is administered at a frequency of 1, 2, 3, or 4 times per day.

In some embodiments, the Compound 1 or a pharmaceutically acceptable salt thereof is administered two times per day.

In some embodiments, the Compound 1 or a pharmaceutically acceptable salt thereof is administered orally.

In some embodiments, the administering results in improvement in liver fibrosis compared to levels before administration of the Compound 1 or a pharmaceutically acceptable salt thereof.

In some embodiments, administration of Compound 1 reduces fat content of the liver.

In some embodiments, administration of Compound 1 reduces fat content of the liver compared to the fat content of the liver prior to the administration of Compound 1 or a pharmaceutically acceptable salt thereof.

In some embodiments, administration of Compound 1 reduces the incidence of or progression of liver cirrhosis.

In some embodiments, administration of Compound 1 reduces the incidence of or progression of liver cirrhosis compared to the incidence of or progression of liver cirrhosis prior to the administration of Compound 1 or a pharmaceutically acceptable salt thereof.

In some embodiments, administration of Compound 1 reduces the incidence of hepatocellular carcinoma.

In some embodiments, administration of Compound 1 reduces the incidence of hepatocellular carcinoma compared to the incidence of hepatocellular carcinoma prior to the administration of Compound 1 or a pharmaceutically acceptable salt thereof.

In some embodiments, administration of Compound 1 reduces the progression of hepatocellular carcinoma.

In some embodiments, administration of Compound 1 decreases in hepatic aminotransferase levels compared to levels before administration of the composition.

In some embodiments, wherein administration of Compound 1 decreases in hepatic aminotransferase levels compared to the hepatic aminotransferase levels prior to the administration of Compound 1 or a pharmaceutically acceptable salt thereof.

In some embodiments, administration of Compound 1 reduces hepatic transaminase compared to levels before treatment.

In some embodiments, wherein administration of Compound 1 reduces the hepatic transaminase levels compared to the hepatic transaminase levels prior to the administration of Compound 1 or a pharmaceutically acceptable salt thereof.

In some embodiments, the hepatic transaminase is alanine transaminase (ALT) or aspartate transaminase (AST) or both.

In some embodiments, administration of Compound 1 reduces hepatic transaminase of about 5% to about 75% compared to the hepatic transaminase levels before treatment.

In some embodiments, administration of Compound 1 reduces hepatic transaminase levels by about 5% to about 75% compared to the hepatic transaminase levels prior to the administration of Compound 1 or a pharmaceutically acceptable salt thereof.

In some embodiments, the hepatic transaminase is alanine transaminase (ALT) or aspartate transaminase (AST) or both.

In some embodiments, administration of Compound 1 reduces alanine aminotransferase (ALT) levels in an individual.

In some embodiments, administration of Compound 1 reduces alanine aminotransferase (ALT) levels in an individual to about 75%, about 70%, about 60%, about 50%, about 40%, about 30%, about 20% or about 10% above normal ALT levels, or at about normal ALT levels.

In some embodiments, administration of Compound 1 reduces alanine aminotransferase (ALT) levels in an individual to about 75%, about 70%, about 60%, about 50%, about 40%, about 30%, about 20%, or to about 10% above normal ALT levels compared to the ALT levels prior to the administration of Compound 1 or a pharmaceutically acceptable salt thereof.

In some embodiments, wherein administration of Compound 1 reduces aspartate aminotransferase (AST) levels in the individual.

In some embodiments, wherein administration of Compound 1 reduces aspartate aminotransferase (AST) levels in an individual to about 75%, about 70%, about 60%, about 50%, about 40%, about 30%, about 20% or about 10% above normal AST levels or at about normal ALT levels.

In some embodiments, administration of Compound 1 reduces aspartate aminotransferase (AST) levels in an individual to about 75%, about 70%, about 60%, about 50%, about 40%, about 30%, about 20%, or to about 10% above normal AST levels compared to the AST levels prior to the administration of Compound 1 or a pharmaceutically acceptable salt thereof.

In some embodiments, the NAFLD is simple steatosis (NAFL).

In some embodiments, the NAFLD is steatohepatitis (NASH).

In some embodiments, the NAFLD is liver cirrhosis.

In some embodiments, the NAFLD is NASH with a degree of fibrosis selected from F1, F2, F3, and F4 fibrosis.

In some embodiments, the NAFLD is NASH with F4 fibrosis.

In some embodiments, the individual has at least one condition selected from hepatic steatosis, lobular inflammation, and hepatocellular ballooning.

In some embodiments, the NAFLD is characterized by a NAFLD activity score (NAS) greater than or equal to 4.

In some embodiments, treating NAFLD is decreasing the NAS at least 1, 2, or 3 points.

In some embodiments, treating NAFLD is decreasing the NAS by at least 1, 2, or 3 points.

In some embodiments, treating NAFLD is reducing the worsening of fibrosis.

In some embodiments, treating NAFLD reduces the worsening or the progression of fibrosis in the individual compared to the fibrosis prior to the administration of Compound 1 or a pharmaceutically acceptable salt thereof.

In some embodiments, treating NAFLD is reversing steatohepatitis.

In some embodiments, treating NAFLD reduced steatohepatitis in the individual compared to the steatohepatitis prior to the administration of Compound 1 or a pharmaceutically acceptable salt thereof.

In some embodiments, treating NAFLD is ceasing progression to F3 or F4 fibrosis.

In some embodiments, treating NAFLD is ceasing progression to F3 or F4 fibrosis in the individual compared to the fibrosis prior to the administration of Compound 1 or a pharmaceutically acceptable salt thereof.

In some embodiments, treating NAFLD is resolving NASH.

In some embodiments, treating NAFLD is resolving NASH in the individual compared to NASH prior to the administration of Compound 1 or a pharmaceutically acceptable salt thereof.

In some embodiments, treating NAFLD is not worsening liver fibrosis.

In some embodiments, treating NAFLD is not worsening liver fibrosis in the individual compared to the liver fibrosis prior to the administration of Compound 1 or a pharmaceutically acceptable salt thereof.

In some embodiments, treating NAFLD is reducing the risk of liver-related death.

In some embodiments, treating NAFLD is improving liver fibrosis by at least one stage.

In some embodiments, treating NAFLD is improving liver fibrosis by at least one stage in the individual compared to the stage of liver fibrosis prior to the administration of Compound 1 or a pharmaceutically acceptable salt thereof.

In some embodiments, treating NAFLD is improving cardiometabolic and/or liver markers.

In some embodiments, treating NAFLD is improving the levels of the cardiometabolic and/or liver markers in the individual compared to the levels of the cardiometabolic and/or liver markers prior to the administration of Compound 1 or a pharmaceutically acceptable salt thereof.

In some embodiments, the individual has been determined to have NALFD using a NAFLD fibrosis score.

In some embodiments, the individual has been determined to have NALFD by a liver biopsy.

In some embodiments, the method further comprising formulating Compound 1 or a pharmaceutically acceptable salt thereof as a tablet or capsule.

In some embodiments, the method further comprises formulating Compound 1 or a pharmaceutically acceptable salt thereof, in a tablet or capsule. In some embodiments, the tablet or capsule comprises a pharmaceutically acceptable carrier.

In some embodiments, further comprises formulating Compound 1 or a pharmaceutically acceptable salt thereof, in a tablet or capsule, wherein the tablet or capsule comprises a pharmaceutically acceptable carrier.

In some embodiments, the administration of the Compound 1 or a pharmaceutically acceptable salt thereof reduces at least ALT, AST, liver fat, or fatty liver index.

In some embodiments, the administration of the Compound 1 or a pharmaceutically acceptable salt thereof reduces at least one of a liver enzyme (e.g., ALT, AST, etc.), liver fat (e.g., determined by ultrasound, etc.), or fatty liver index.

In some embodiments, the individual has no fibrosis and no inflammation of the liver. In some embodiments, the individual has no fibrosis. In some embodiments, the individual has limited fibrosis. In some embodiments, the individual has intermediate fibrosis. In some embodiments, the individual has advanced fibrosis. In some embodiments, the individual has cirrhosis.

In some embodiments, the individual has stage F0 fibrosis. In some embodiments, the individual has stage F1 fibrosis. In some embodiments, the individual has stage F2 fibrosis. In some embodiments, the individual has stage F3 fibrosis. In some embodiments, the individual has stage F4 fibrosis. In some embodiments, the individual has stage F0-F1 fibrosis. In some embodiments, the individual has stage F1-F2 fibrosis. In some embodiments, the individual has stage F2-F3 fibrosis. In some embodiments, the individual has stage F3-F4 fibrosis. In some embodiments, the individual has stage F0-F2 fibrosis. In some embodiments, the individual has stage F0-F3 fibrosis. In some embodiments, the individual has stage F1-F3 fibrosis. In some embodiments, the individual has stage F1-F4 fibrosis. In some embodiments, the individual has stage F2-F4 fibrosis.

NAFLD fibrosis score is a validated scoring system comprised of six routinely measured parameters (i.e., age, hyperglycemia, BMI, platelet counts, albumin, and AST/ALT ratio) which can be used to identify NAFLD patients likely to have F3-F4 fibrosis.

### Crystalline Forms of Compound 1

The crystalline forms and processes useful in the preparation of the crystalline forms of Compound 1 are described in WO2010/135505. The three different forms are labeled as Form A-1, Form A-IV, and Form A-VI.

### Compound 1, Form A-I (Anhydrous)

One aspect of the present invention relates to anhydrous Form A-I of 4-[6-(6-Methanesulfonyl-2-methyl-pyridin-3-ylamino)-5-methoxy-pyrimidin-4-yloxy]-piperidine-1-carboxylic acid isopropyl ester (Compound 1). Peaks, in terms of 2 θ, from a powder X-ray diffraction pattern from a representative sample of Form A-I are provided below in TABLE 1.

TABLE 1
(Compound 1, Form A-I)

| Pos. | d-spacing | Rel. Int. |
|---|---|---|
| [°2θ] | [A] | [%] |
| 8.0 | 11.0 | 100.0 |
| 12.1 | 7.3 | 10.0 |
| 13.6 | 6.5 | 12.0 |
| 13.8 | 6.4 | 10.0 |
| 15.2 | 5.8 | 10.0 |
| 15.6 | 5.7 | 9.0 |
| 16.2 | 5.5 | 20.0 |
| 16.4 | 5.4 | 51.0 |
| 17.2 | 5.1 | 11.0 |
| 17.7 | 5.0 | 18.0 |
| 19.8 | 4.5 | 10.0 |
| 21.2 | 4.2 | 23.0 |
| 22.6 | 3.9 | 14.0 |
| 22.9 | 3.9 | 9.0 |
| 24.5 | 3.6 | 18.0 |

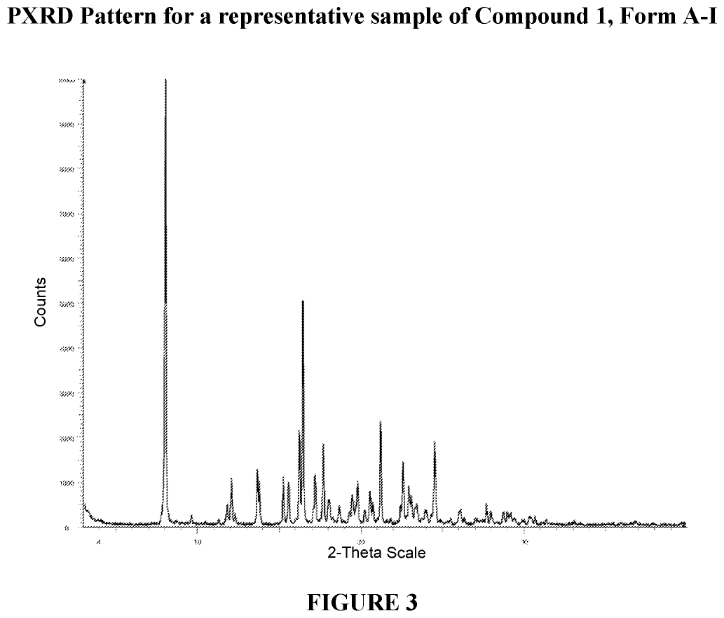

In some embodiments, Compound 1 is anhydrous Form A-I. In some embodiments, Compound 1 is Form A-I and has a powder X-ray diffraction pattern comprising peaks, in terms of 2 θ, at 8.0°±0.2°, and 17.7°±0.2°. In some embodiments, Compound 1 is Form A-I and has a powder X-ray diffraction pattern comprising peaks, in terms of 2 θ, at 8.0°±0.2°, 16.4°±0.2°, and 17.7°±0.2°. In some embodiments, Compound 1 is Form A-I and has a powder X-ray diffraction pattern comprising peaks, in terms of 2 θ, at 8.0°±0.2°, 16.4°±0.2°, 17.7°±0.2°, and 21.2°±0.2°. In some embodiments, Compound 1 is Form A-I and has a powder X-ray diffraction pattern comprising peaks, in terms of 2 θ, at 8.0°±0.2°, 16.4°±0.2°, 17.7°±0.2°, 21.2°±0.2°, and 24.5°±0.2°. In some embodiments, Compound 1 is Form A-I and has a powder X-ray diffraction pattern comprising peaks, in terms of 2 θ, at 8.0°±0.2°, 12.1°±0.2°, 13.6°±0.2°, 13.8°±0.2°, 15.2°±0.2°, 15.6°±0.2°, 16.2°±0.2°, 16.4°±0.2°, 17.2°±0.2°, 17.7°±0.2°, 19.8°±0.2°, 21.2°±0.2°, 22.6°±0.2°, 22.9°±0.2°, and 24.5°±0.2°. In some embodiments, Compound 1 is Form A-I and has a powder X-ray diffraction pattern substantially as shown in FIG. 3. In some embodiments, Compound 1 is Form A-I and has a differential scanning calorimetry trace comprising an endotherm with an extrapolated onset temperature of melting of 164.6° C., a peak temperature of melting of 166.8° C. and a heat of melting of 86.2 J/g at a scan rate of 10° C./minute. In some embodiments, Compound 1 is Form A-I and has a thermogravimetric analysis profile showing about 0.3% weight loss from ambient temperature up to 165° C. and including the melting of the sample. These results indicate that crystalline Form A-I is an anhydrous form.

### Compound 1, Form A-IV (Anhydrous)

One aspect of the present invention relates to anhydrous Form A-IV of 4-[6-(6-Methanesulfonyl-2-methyl-pyridin-3-ylamino)-5-methoxy-pyrimidin-4-yloxy]-piperidine-1-carboxylic acid isopropyl ester (Compound 1). Peaks, in terms of 2θ, from a powder X-ray diffraction pattern from a representative sample of Form A-IV are provided below in TABLE 2.

TABLE 2
(Compound 1, Form A-IV)

| Pos. | d-spacing | Rel. Int. |
|---|---|---|
| [°2θ] | [A] | [%] |
| 9.0 | 9.8 | 10.0 |
| 9.5 | 9.3 | 52.0 |
| 11.3 | 7.8 | 11.0 |
| 13.5 | 6.5 | 14.0 |
| 13.6 | 6.5 | 25.0 |
| 14.1 | 6.3 | 10.0 |
| 14.8 | 6.0 | 18.0 |
| 16.5 | 5.4 | 62.0 |
| 18.2 | 4.9 | 83.0 |
| 18.5 | 4.8 | 14.0 |
| 19.1 | 4.6 | 26.0 |
| 19.5 | 4.6 | 43.0 |
| 19.9 | 4.5 | 100.0 |
| 20.0 | 4.4 | 93.0 |
| 20.9 | 4.2 | 26.0 |
| 21.2 | 4.2 | 31.0 |
| 22.9 | 3.9 | 16.0 |
| 23.4 | 3.8 | 26.0 |
| 24.9 | 3.6 | 27.0 |
| 25.5 | 3.5 | 11.0 |
| 27.0 | 3.3 | 11.0 |
| 27.3 | 3.3 | 36.0 |
| 27.7 | 3.2 | 42.0 |
| 29.7 | 3.0 | 10.0 |
| 31.4 | 2.8 | 11.0 |

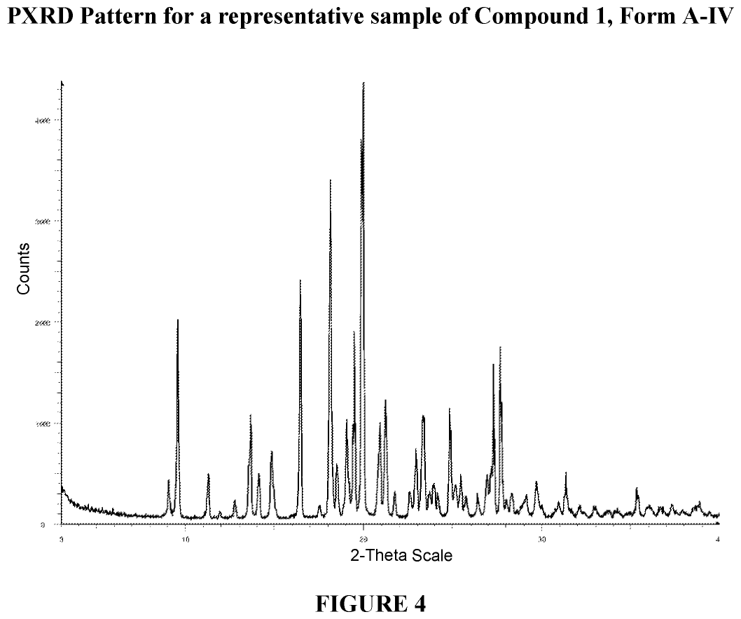

In some embodiments, Compound 1 is anhydrous Form A-IV. In some embodiments, Compound 1 is Form A-IV and has a powder X-ray diffraction pattern comprising peaks, in terms of 2 θ, at 19.9°±0.2°. In some embodiments, Compound 1 is Form A-IV and has a powder X-ray diffraction pattern comprising peaks, in terms of 2 θ, at 18.20±0.2°, and 19.9°±0.2°. In some embodiments, Compound 1 is Form A-IV and has a powder X-ray diffraction pattern comprising peaks, in terms of 2 θ, at 16.5°±0.2°, 18.2°±0.2°, and 19.9°±0.2°. In some embodiments, Compound 1 is Form A-IV and has a powder X-ray diffraction pattern comprising peaks, in terms of 2 θ, at 9.5°±0.2°, 16.5°±0.2°, 18.2°±0.2°, and 19.9°±0.2°. In some embodiments, Compound 1 is Form A-IV and has a powder X-ray diffraction pattern comprising peaks, in terms of 2 θ, at 9.5°±0.2°, 16.5°±0.2°, 18.2°±0.2°, 19.9°±0.2° and 23.4°±0.2°. In some embodiments, Compound 1 is Form A-IV and has a powder X-ray diffraction pattern comprising peaks, in terms of 2 θ, at 9.0°±0.2°, 9.5°±0.2°, 11.3°±0.2°, 13.5°±0.2°, 13.6°±0.2°, 14.1°±0.2°, 14.8°±0.2°, 16.5°±0.2°, 18.2°±0.2°, 18.5°±0.2°, 19.1°±0.2°, 19.5°±0.2°, 19.9°±0.2°, 20.0°±0.2°, 20.9°±0.2°, 21.2°±0.2°, 22.9°±0.2°, 23.4°±0.2°, 24.9°±0.2°, 25.5°±0.2°, 27.0°±0.2°, 27.3°±0.2°, 27.7°±0.2°, 29.7°±0.2°, and 31.4°±0.2°. In some embodiments, Compound 1 is Form A-IV and has a powder X-ray diffraction pattern substantially as shown in FIG. 4. In some embodiments, Compound 1 is Form A-IV and has a differential scanning calorimetry trace comprising an endotherm with an extrapolated onset temperature of melting of 154.1° C., a peak temperature of melting of 155.6° C. and a heat of melting of 86.8 J/g at a scan rate of 10° C./minute. In some embodiments, Compound 1 is Form A-IV and has a thermogravimetric analysis profile showing about 0.1% weight loss from ambient temperature up to 165° C. and including the melting of the sample. These results indicate that crystalline Form A-IV is an anhydrous form.

### Compound 1, Form A-VI (Anhydrous)

One aspect of the present invention relates to anhydrous Form A-VI of 4-[6-(6-Methanesulfonyl-2-methyl-pyridin-3-ylamino)-5-methoxy-pyrimidin-4-yloxy]-piperidine-1-carboxylic acid isopropyl ester (Compound 1). Peaks, in terms of 2 θ, from a powder X-ray diffraction pattern from a representative sample of Form A-VI are provided below in TABLE 3.

TABLE 3
(Compound 1, Form A-VI)

| Pos. | d-spacing | Rel. Int. |
|---|---|---|
| [°2θ] | [A] | [%] |
| 5.8 | 15.1 | 100.0 |
| 13.2 | 6.7 | 1.0 |
| 13.6 | 6.5 | 1.0 |
| 14.6 | 6.1 | 10.0 |
| 14.9 | 5.9 | 1.0 |
| 16.4 | 5.4 | 1.0 |
| 17.6 | 5.0 | 1.0 |
| 18.3 | 4.9 | 2.0 |
| 18.9 | 4.7 | 18.0 |
| 19.7 | 4.5 | 2.0 |
| 21.5 | 4.1 | 1.0 |
| 22.2 | 4.0 | 5.0 |
| 22.7 | 3.9 | 3.0 |
| 23.5 | 3.8 | 19.0 |
| 24.0 | 3.7 | 3.0 |
| 24.8 | 3.6 | 1.0 |
| 25.1 | 3.6 | 1.0 |

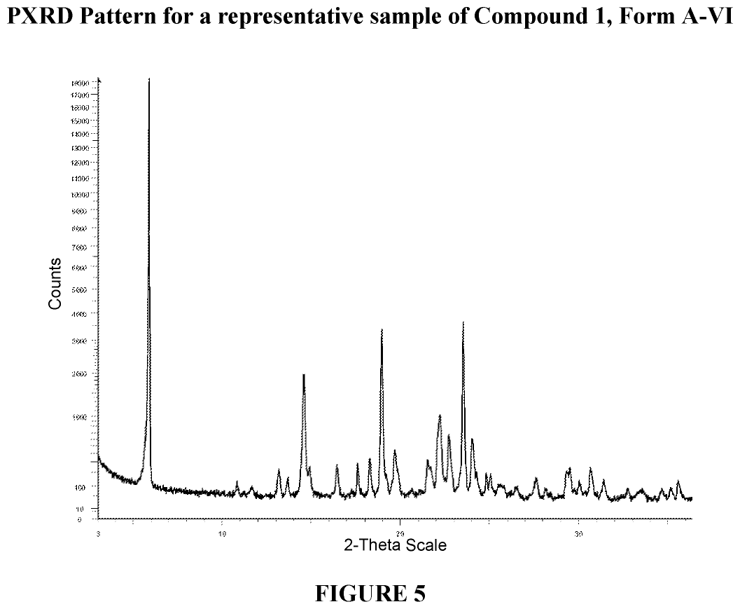

In some embodiments, Compound 1 is anhydrous Form A-VI. In some embodiments, Compound 1 is Form A-VI and has a powder X-ray diffraction pattern comprising peaks, in terms of 2 θ, at 5.8°±0.2°. In some embodiments, Compound 1 is Form A-VI and has a powder X-ray diffraction pattern comprising peaks, in terms of 2 θ, at 5.8°±0.2°, and 23.5°±0.2°. In some embodiments, Compound 1 is Form A-VI and has a powder X-ray diffraction pattern comprising peaks, in terms of 2 θ, at 5.8°±0.2°, 18.9°±0.2°, and 23.5°±0.2°. In some embodiments, Compound 1 is Form A-VI and has a powder X-ray diffraction pattern comprising peaks, in terms of 2 θ, at 5.8°±0.2°, 14.6°±0.2°, 18.9°±0.2°, and 23.5°±0.2°. In some embodiments, Compound 1 is Form A-VI and has a powder X-ray diffraction pattern comprising peaks, in terms of 2 θ, at 5.8°±0.2°, 14.6°±0.2°, 18.9°±0.2°, 22.2°±0.2°, and 23.5°±0.2°. In some embodiments, Compound 1 is Form A-VI and has a powder X-ray diffraction pattern comprising peaks, in terms of 2 θ, at 5.8°±0.2°, 13.2°±0.2, 13.6°±0.20, 14.6°±0.20, 14.9°±0.2°, 16.4°±0.2°, 17.6°±0.2, 18.3°±0.20, 18.9°±0.2°, 19.7°±0.2°, 21.5°±0.2°, 22.2°±0.2°, 22.7°±0.2°, 23.5°±0.2°, 24.0°±0.2°, 24.8°±0.2°, and 25.1°±0.2°. In some embodiments, Compound 1 is Form A-VI and has a powder X-ray diffraction pattern substantially as shown in FIG. 5. In some embodiments, Compound 1 is Form A-VI and has a differential scanning calorimetry trace comprising an endotherm with an extrapolated onset temperature of melting of 162.1° C., a peak temperature of melting of 164.0° C. and a heat of melting of 92.2 J/g at a scan rate of 10° C./minute. In some embodiments, Compound 1 is Form A-VI and has a thermogravimetric analysis profile showing about 0.2% weight loss from ambient temperature up to 165° C. and including the melting of the sample. These results indicate that crystalline Form A-VI is an anhydrous form.

### Pharmaceutical Compositions

A further aspect of the present invention pertains to pharmaceutical compositions comprising one or more compounds as described herein and one or more pharmaceutically acceptable carriers.

Some embodiments pertain to pharmaceutical compositions comprising a compound of the present invention and a pharmaceutically acceptable carrier.

Some embodiments of the present invention include a method of producing a pharmaceutical composition comprising admixing at least one compound according to any of the compound embodiments disclosed herein and a pharmaceutically acceptable carrier.

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound of the invention in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

While it is possible that, for use in the prophylaxis or treatment, a compound of the invention may, in an alternative use, be administered as a raw or pure chemical, it is preferable however to present the compound or active ingredient as a pharmaceutical formulation or composition further comprising a pharmaceutically acceptable carrier.

The invention thus further provides pharmaceutical formulations comprising a compound of the invention or a pharmaceutically acceptable salt, solvate, hydrate or derivative thereof together with one or more pharmaceutically acceptable carriers thereof and/or prophylactic ingredients. The carrier(s) must be “acceptable” in the sense of being compatible with the other ingredients of the formulation and not overly deleterious to the recipient thereof.

Compounds of the present invention or a salt, solvate, hydrate or physiologically functional derivative thereof can be used as active ingredients in pharmaceutical compositions, specifically as S1P1 receptor modulators. The term “active ingredient” is defined in the context of a “pharmaceutical composition” and is intended to mean a component of a pharmaceutical composition that provides the primary pharmacological effect, as opposed to an “inactive ingredient” which would generally be recognized as providing no pharmaceutical benefit.

The dose when using the compounds of the present invention can vary and as is customary and known to the physician, it is to be tailored to the individual conditions in each individual case. It depends, for example, on the nature and severity of the illness to be treated, on the condition of the individual, or on whether an acute or chronic disease state is treated or prophylaxis is conducted or on whether further active compounds are administered in addition to the compounds of the present invention. Representative doses of the present invention include, but are not limited to, about 1 mg to about 1000 mg. In some embodiments, the dose is, or is about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 125, 130, 140, 150, 160, 170, 175, 180, 190, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 mg. Multiple doses may be administered during the day, especially when relatively large amounts are deemed to be needed, for example 2, 3 or 4 doses. Depending on the individual and as deemed appropriate by the individual's physician or caregiver it may be necessary to deviate upward or downward from the doses described herein.

The amount of active ingredient or an active salt, solvate or hydrate derivative thereof, required for use in treatment will vary not only with the particular salt selected but also with the route of administration, the nature of the condition being treated and the age and condition of the individual and will ultimately be at the discretion of the attendant physician or clinician. Representative factors include the type, age, weight, sex, diet and medical condition of the individual, the severity of the disease, the route of administration, pharmacological considerations such as the activity, efficacy, pharmacokinetic and toxicology profiles of the particular compound employed, whether a drug delivery system is utilized, whether an acute or chronic disease state is being treated or prophylaxis is conducted or whether further active compounds are administered in addition to the compounds of the present invention and as part of a drug combination. The dosage regimen for treating a disease condition with the compounds and/or compositions of this invention is selected in accordance with a variety factors including those cited above. Thus, the actual dosage regimen employed may vary widely and therefore may deviate from a preferred dosage regimen and one skilled in the art will recognize that dosage and dosage regimens outside these typical ranges can be tested and, where appropriate, may be used in the methods of this invention.

The desired dose may conveniently be presented in a single dose or as divided doses administered at appropriate intervals, for example, as 2, 3, 4 or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

In some embodiments, the compositions are tablets or capsules for oral administration.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed by Berge et al., Journal of Pharmaceutical Sciences, 66:1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

Compounds of the present invention can be converted to “pro-drugs.” The term “pro-drugs” refers to compounds that have been modified with specific chemical groups known in the art and that when administered into an individual undergo biotransformation to give the parent compound. Pro-drugs can thus be viewed as compounds of the invention containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987, both of which are hereby incorporated by reference in their entirety.

As will be recognized, the steps of the methods of the present invention need not be performed any particular number of times or in any particular sequence. Additional objects, advantages and novel features of this invention will become apparent to those skilled in the art upon examination of the following examples thereof, which are intended to be illustrative and not intended to be limiting.

### EXAMPLES

### Example 1: Preparation of Compound 1 (4-[6-(6-Methanesulfonyl-2-methyl-pyridin-3-ylamino)-5-methoxy-pyrimidin-4-yloxy]-piperidine-1-carboxylic acid isopropyl ester) and Crystalline Forms Thereof

The preparation of Compound 1 (4-[6-(6-Methanesulfonyl-2-methyl-pyridin-3-ylamino)-5-methoxy-pyrimidin-4-yloxy]-piperidine-1-carboxylic acid isopropyl ester) is described in International Patent Application No. PCT/US2006/000567, published as International Publication No. WO2006/083491 (see Compound 84); U.S. Ser. No. 11/327,896, published as US publication US2007/0167473; International Patent Application No. PCT/US2009/062606, published as International Publication No. WO2010/059384; International Patent Application No. PCT/US2009/062610, published as International Publication No. WO2010/059385; and International Patent Application No. PCT/US2010/035538, published as International Publication No. WO 2010/135506; the entire contents of each are incorporated herein by reference in their entirety.

The preparations of crystalline forms of Compound 1 are described in International Patent Application No. PCT/US2010/035536, published as International Publication No. WO 2010/135505, the entire contents incorporated herein by reference in their entirety.

### Example 2: Oral Glucose Tolerance Test (oGTT) with Compound 1

Animals were grouped in regular cages (rats 2/cage, mice 4/cage) with bedding under normal light conditions (lights on 6:30 am-6:30 pm). They were given ad libitum access to food and water. Animals were allowed to acclimate to the facility for several days before handling.

Procedures:

Mouse: Following 2 handling sessions, animals were fasted for 3-16 h.

The oral glucose tolerance test (oGTT) was executed as follows: Compound was administered 0-30 min prior to first blood sample. At time 0, a tail nick and glucose test was performed with a hand-held glucometer, then a bolus of glucose (2 mg/kg, p.o.) was administered. Glucose was again tested at 20, 40, 60, and 120 minutes post glucose administration. During the entire oGTT, a total volume of approximately 10 drops of blood was collected.

Rat: Animals are fasted 3-16 h.

The oral glucose tolerance test (OGTT) was executed as follows: At time minus 30 min, blood was collected via a tail nick and a glucose test performed with a hand-held glucometer, and compound was then administered. At time 0, blood was again collected for glucose reading and then a bolus of glucose (3 g/kg p.o., 6 mL/kg) administered. Blood glucose levels were further tested at 30, 60, and 120 min post glucose administration. During the entire oGTT, a total volume of approximately 8 drops of blood was collected. Rats were used twice with a one week lapse between experiments.

### Example 3: Powder X-Ray Diffraction Patterns (PXRD) for Form A-I, Form A-IV, and Form A-VI for Compound 1

The crystalline forms of Compound 1 were characterized as to their powder X-ray diffraction patterns (PXRD), for example as follows. The sample was examined using an x-ray diffractometer (Bruker AXS Model 08 Advance) equipped with Gobel mirror incident beam and PSD detector (type lynxEye). The sample was placed on to zero-background holder and scanned under ambient conditions of temperature and humidity. The sample was scanned from 3 to 40° 2 θ at a step size of 0.019° 2 θ and a time per step of 38.4 seconds. The radiation was CuKα (45 KkV and 40 mA). The divergence slit and anti-scatter slit were 0.982° and 0.499°, respectively. One skilled in the art will recognize that the PXRD measured values which follow herein (°2 θ, FWHM, d-spacing and % Relative Intensity) will vary with various parameters including, but not limited to, precision and method of grinding during sample preparation, crystal size and morphology, diffractometer configuration, and data collection parameters/experimental conditions. One skilled in the art will further recognize that the crystal forms of the present invention are not limited to crystalline forms which provide a powder X-ray diffraction pattern, and/or peak characteristics identical to those described in the Tables and Figures which follow herein. Notwithstanding, one skilled in the art will recognize that any crystalline forms of Compound 1 which provide a powder x-ray diffraction pattern and/or peak characteristics which are substantially similar to those described in the Tables and Figures which follow herein, shall fall within the scope of this invention. See FIG. 3 (Compound 1, Form A-I), FIG. 4 (Compound 1, Form A-IV), and FIG. 5 (Compound 1, Form A-VI).

### Example 4: Differential Scanning Calorimetry (DSC) for Form A-I, Form A-IV, and Form A-VI for Compound 1

The crystalline forms of the present invention were subjected to DSC analysis. A representative sample was tested using a TA Instruments DSC Q100 differential scanning calorimeter. The sample was analyzed as received in a crimped TA Instrument aluminum sample pan and was program heated from ambient to 250° C. at 10° C./min under nitrogen purge.

Compound 1, Form A-I was observed to have a differential scanning calorimetry trace comprising an endotherm with an extrapolated onset temperature of melting of 164.6° C., a peak temperature of melting of 166.8° C. and a heat of melting of 86.2 J/g.

Compound 1, Form A-IV was observed to have a differential scanning calorimetry trace comprising an endotherm with an extrapolated onset temperature of melting of 154.1° C., a peak temperature of melting of 155.6° C. and a heat of melting of 86.8 J/g.

Compound 1, Form A-VI was observed to have a differential scanning calorimetry trace comprising an endotherm with an extrapolated onset temperature of melting of 162.1° C., a peak temperature of melting of 164.0° C. and a heat of melting of 92.2 J/g.

### Example 5: Thermogravimetric Analysis (TGA) for Form A-I, Form A-IV, and Form A-VI for Compound 1

The crystalline forms of the present invention were subjected to DSC analysis. A representative sample was tested for weight loss using a TA Instruments TGA Q50 thermogravimetric calorimeter. The sample was analyzed as received and was program heated from ambient to 300° C. at 10° C./min under nitrogen purge.

Compound 1, Form A-I was observed to have a thermogravimetric analysis profile showing about 0.3% weight loss from ambient temperature up to 165° C. and including the melting of the sample. These results indicate that crystalline Form A-I is an anhydrous form.

Compound 1, Form A-IV was observed to have a thermogravimetric analysis profile showing about 0.1% weight loss from ambient temperature up to 165° C. and including the melting of the sample. These results indicate that crystalline Form A-IV is an anhydrous form.

Compound 1, Form A-VI was observed to have a thermogravimetric analysis profile showing about 0.2% weight loss from ambient temperature up to 165° C. and including the melting of the sample. These results indicate that crystalline Form A-VI is an anhydrous form.

### Example 6: Treatment of Compound 1 and MBX-2982 in a Model of Non-Alcoholic Fatty Liver Disease (NAFLD)

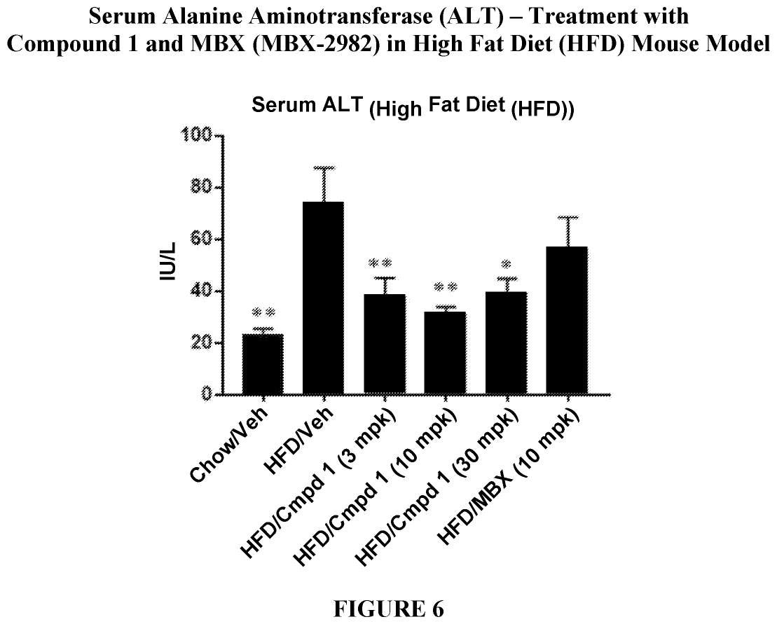

The effects of treatment with a Compound 1 were evaluated in a murine model of non-alcoholic fatty liver disease. Male C57BL/6 mice (8 weeks) were fed either normal or high fat diet (HFD) (60% TD06414, Envigo) and given water ad libitum. Six weeks after diet initiation, animals were treated with one of the following five times per week for six consecutive weeks: 1) normal diet (negative control) and vehicle (60% PEG 400); 2) high fat diet and vehicle; 3) high fat diet and 3 mg/kg/day Compound 1; 4) high fat diet and 10 mg/kg/day Compound 1; 5) high fat diet and 30 mg/kg/day Compound 1; and 6) high fat diet and 10 mg/kg/day MBX-2982 (a GPR119 agonist). At 20 weeks, mice were sacrificed and serum plasma was collected for measurement of various biochemical parameters, including serum transaminases. Increases in serum ALT (a known marker of hepatocellular injury) were observed in all animals exposed to the high fat diet. These increases were blocked by treatment with Compound 1, but not MBX-2982 (see FIG. 6).

While the invention has been described in conjunction with the detailed description thereof, the foregoing description is intended to illustrate and not limit the scope of the invention, which is defined by the scope of the appended claims. Those skilled in the art will recognize that various modifications, additions, substitutions, and variations to the illustrative examples set forth herein can be made without departing from the spirit of the invention and are, therefore, considered within the scope of the invention. Other aspects, advantages, and modifications are within the scope of the following claims.

What is claimed is:

1. A method of treating non-alcoholic fatty liver disease (NAFLD) or a condition related thereto in an individual in need thereof comprising administering a therapeutically effective amount of 4-[6-(6-Methanesulfonyl-2-methyl-pyridin-3-ylamino)-5-methoxy-pyrimidin-4-yloxy]-piperidine-1-carboxylic acid isopropyl ester (Compound 1), or a pharmaceutically acceptable salt, hydrate, or solvate thereof.

2. A compound selected from the following compound and pharmaceutically acceptable salts, solvates, and hydrates thereof:
4-[6-(6-Methanesulfonyl-2-methyl-pyridin-3-ylamino)-5-methoxy-pyrimidin-4-yloxy]-piperidine-1-carboxylic acid isopropyl ester (Compound 1),
for use in a method of treating non-alcoholic fatty liver disease (NAFLD) or a condition related thereto in an individual comprising administering a therapeutically effective amount of Compound 1, or a pharmaceutically acceptable salt, hydrate, or solvate thereof.

3. Use of a compound selected from the following compound and pharmaceutically acceptable salts, solvates, and hydrates thereof:
4-[6-(6-Methanesulfonyl-2-methyl-pyridin-3-ylamino)-5-methoxy-pyrimidin-4-yloxy]-piperidine-1-carboxylic acid isopropyl ester (Compound 1),
in the manufacture of a medicament for the treatment of non-alcoholic fatty liver disease (NAFLD) or a condition related thereto in an individual comprising administering a therapeutically effective amount of Compound 1, or a pharmaceutically acceptable salt, hydrate, or solvate thereof.

4. The method according to claim 1, the compound according to claim 2, or the use according to claim 3, wherein the daily therapeutically effective amount of Compound 1 or the pharmaceutically acceptable salt thereof is about 2.5 mg to 800 mg.

5. The method according to claim 1, the compound according to claim 2, or the use according to claim 3, wherein the daily therapeutically effective amount of Compound 1 or the pharmaceutically acceptable salt thereof is about 100 mg to 800 mg.

6. The method according to any one of claims 1, 4, and 5; the compound according to any one of claims 2, 4, and 5; or the use according to any one of claims 3 to 5; wherein the Compound 1 or the pharmaceutically acceptable salt thereof is administered at a frequency of 1, 2, 3, or 4 times per day.

7. The method according to any one of claims 1 and 4 to 6; the compound according to any one of claims 2 and 4 to 6; or the use according to any one of claims 3 to 6; wherein the Compound 1 or the pharmaceutically acceptable salt thereof is administered two times per day.

8. The method according to any one of claims 1 and 4 to 7; the compound according to any one of claims 2 and 4 to 7; or the use according to any one of claims 3 to 7; wherein the Compound 1 or a pharmaceutically acceptable salt thereof is administered orally.

9. The method according to any one of claims 1 and 4 to 8; the compound according to any one of claims 2 and 4 to 8; or the use according to any one of claims 3 to 8; wherein the administering results in improvement in liver fibrosis compared to levels before administration of the Compound 1 or a pharmaceutically acceptable salt thereof.

10. The method according to any one of claims 1 and 4 to 8; the compound according to any one of claims 2 and 4 to 8; or the use according to any one of claims 3 to 8; wherein administration of Compound 1 reduces fat content of the liver compared to the fat content of the liver prior to the administration of Compound 1 or a pharmaceutically acceptable salt thereof.

11. The method according to any one of claims 1 and 4 to 8; the compound according to any one of claims 2 and 4 to 8; or the use according to any one of claims 3 to 8; wherein administration of Compound 1 reduces the incidence of or progression of liver cirrhosis.

12. The method according to any one of claims 1 and 4 to 8; the compound according to any one of claims 2 and 4 to 8; or the use according to any one of claims 3 to 8; wherein administration of Compound 1 reduces the incidence of hepatocellular carcinoma.

13. The method according to any one of claims 1 and 4 to 8; the compound according to any one of claims 2 and 4 to 8; or the use according to any one of claims 3 to 8; wherein administration of Compound 1 reduces the progression of hepatocellular carcinoma.

14. The method according to any one of claims 1 and 4 to 8; the compound according to any one of claims 2 and 4 to 8; or the use according to any one of claims 3 to 8; wherein administration of Compound 1 decreases in hepatic aminotransferase levels compared to the hepatic aminotransferase levels prior to the administration of Compound 1 or a pharmaceutically acceptable salt thereof.

15. The method according to any one of claims 1 and 4 to 8; the compound according to any one of claims 2 and 4 to 8; or the use according to any one of claims 3 to 8; wherein administration of Compound 1 reduces the hepatic transaminase levels compared to the hepatic transaminase levels prior to the administration of Compound 1 or a pharmaceutically acceptable salt thereof.

16. The method according to any one of claims 1 and 4 to 8; the compound according to any one of claims 2 and 4 to 8; or the use according to any one of claims 3 to 8; wherein administration of Compound 1 reduces hepatic transaminase levels by about 5% to about 75% compared to the hepatic transaminase levels prior to the administration of Compound 1 or a pharmaceutically acceptable salt thereof.

17. The method according to claim 15 or 16, wherein the hepatic transaminase is alanine transaminase (ALT) or aspartate transaminase (AST) or both.

18. The method according to any one of claims 1 and 4 to 8; the compound according to any one of claims 2 and 4 to 8; or the use according to any one of claims 3 to 8; wherein administration of Compound 1 reduces alanine aminotransferase (ALT) levels in an individual.

19. The method according to any one of claims 1 and 4 to 8; the compound according to any one of claims 2 and 4 to 8; or the use according to any one of claims 3 to 8; wherein administration of Compound 1 reduces alanine aminotransferase (ALT) levels in an individual to about 75%, about 70%, about 60%, about 50%, about 40%, about 30%, about 20% or about 10% above normal ALT levels, or at about normal ALT levels.

20. The method according to any one of claims 1 and 4 to 8; the compound according to any one of claims 2 and 4 to 8; or the use according to any one of claims 3 to 8; wherein administration of Compound 1 reduces alanine aminotransferase (ALT) levels in an individual to about 75%, about 70%, about 60%, about 50%, about 40%, about 30%, about 20%, or to about 10% above normal ALT levels compared to the ALT levels prior to the administration of Compound 1 or a pharmaceutically acceptable salt thereof.

21. The method according to any one of claims 1 and 4 to 8; the compound according to any one of claims 2 and 4 to 8; or the use according to any one of claims 3 to 8; wherein administration of Compound 1 reduces aspartate aminotransferase (AST) levels in the individual.

22. The method according to any one of claims 1 and 4 to 8; the compound according to any one of claims 2 and 4 to 8; or the use according to any one of claims 3 to 8; wherein administration of Compound 1 reduces aspartate aminotransferase (AST) levels in an individual to about 75%, about 70%, about 60%, about 50%, about 40%, about 30%, about 20% or about 10% above normal AST levels or at about normal ALT levels.

23. The method according to any one of claims 1 and 4 to 8; the compound according to any one of claims 2 and 4 to 8; or the use according to any one of claims 3 to 8; wherein administration of Compound 1 reduces aspartate aminotransferase (AST) levels in an individual to about 75%, about 70%, about 60%, about 50%, about 40%, about 30%, about 20%, or to about 10% above normal AST levels compared to the AST levels prior to the administration of Compound 1 or a pharmaceutically acceptable salt thereof.

24. The method according to any one of claims 1 and 4 to 8; the compound according to any one of claims 2 and 4 to 8; or the use according to any one of claims 3 to 8; wherein the NAFLD is simple steatosis (NAFL).

25. The method according to any one of claims 1 and 4 to 8; the compound according to any one of claims 2 and 4 to 8; or the use according to any one of claims 3 to 8; wherein the NAFLD is steatohepatitis (NASH).

26. The method according to any one of claims 1 and 4 to 8; the compound according to any one of claims 2 and 4 to 8; or the use according to any one of claims 3 to 8; wherein the NAFLD is liver cirrhosis.

27. The method according to any one of claims 1 and 4 to 8; the compound according to any one of claims 2 and 4 to 8; or the use according to any one of claims 3 to 8; wherein the NAFLD is NASH with a degree of fibrosis selected from F1, F2, F3, and F4 fibrosis.

28. The method according to any one of claims 1 and 4 to 8; the compound according to any one of claims 2 and 4 to 8; or the use according to any one of claims 3 to 8; wherein the NAFLD is NASH with F4 fibrosis.

29. The method according to any one of claims 1 and 4 to 8; the compound according to any one of claims 2 and 4 to 8; or the use according to any one of claims 3 to 8; wherein the individual has at least one condition selected from hepatic steatosis, lobular inflammation, and hepatocellular ballooning.

30. The method according to any one of claims 1 and 4 to 8; the compound according to any one of claims 2 and 4 to 8; or the use according to any one of claims 3 to 8; wherein the NAFLD is characterized by a NAFLD activity score (NAS) greater than or equal to 4.

31. The method according to any one of claims 1 and 4 to 8; the compound according to any one of claims 2 and 4 to 8; or the use according to any one of claims 3 to 8; wherein treating NAFLD is decreasing the NAS by at least 1, 2, or 3 points.

32. The method according to any one of claims 1 and 4 to 8; the compound according to any one of claims 2 and 4 to 8; or the use according to any one of claims 3 to 8; wherein treating NAFLD reduces the worsening or the progression of fibrosis in the individual compared to the fibrosis prior to the administration of Compound 1 or a pharmaceutically acceptable salt thereof.

33. The method according to any one of claims 1 and 4 to 8; the compound according to any one of claims 2 and 4 to 8; or the use according to any one of claims 3 to 8; wherein treating NAFLD reduced steatohepatitis in the individual compared to the steatohepatitis prior to the administration of Compound 1 or a pharmaceutically acceptable salt thereof.

34. The method according to any one of claims 1 and 4 to 8; the compound according to any one of claims 2 and 4 to 8; or the use according to any one of claims 3 to 8; wherein treating NAFLD is ceasing progression to F3 or F4 fibrosis in the individual compared to the fibrosis prior to the administration of Compound 1 or a pharmaceutically acceptable salt thereof.

35. The method according to any one of claims 1 and 4 to 8; the compound according to any one of claims 2 and 4 to 8; or the use according to any one of claims 3 to 8; wherein treating NAFLD is resolving NASH in the individual compared to NASH prior to the administration of Compound 1 or a pharmaceutically acceptable salt thereof.

36. The method according to any one of claims 1 and 4 to 8; the compound according to any one of claims 2 and 4 to 8; or the use according to any one of claims 3 to 8; wherein treating NAFLD is not worsening liver fibrosis in the individual compared to the liver fibrosis prior to the administration of Compound 1 or a pharmaceutically acceptable salt thereof.

37. The method according to any one of claims 1 and 4 to 8; the compound according to any one of claims 2 and 4 to 8; or the use according to any one of claims 3 to 8; wherein treating NAFLD is reducing the risk of liver-related death.

38. The method according to any one of claims 1 and 4 to 8; the compound according to any one of claims 2 and 4 to 8; or the use according to any one of claims 3 to 8; wherein treating NAFLD is improving liver fibrosis by at least one stage in the individual compared to the stage of liver fibrosis prior to the administration of Compound 1 or a pharmaceutically acceptable salt thereof.

39. The method according to any one of claims 1 and 4 to 8; the compound according to any one of claims 2 and 4 to 8; or the use according to any one of claims 3 to 8; wherein treating NAFLD is improving levels of the cardiometabolic and/or liver markers in the individual compared to the levels of the cardiometabolic and/or liver markers prior to the administration of Compound 1 or a pharmaceutically acceptable salt thereof.

40. The method according to any one of claims 1 and 4 to 8; the compound according to any one of claims 2 and 4 to 8; or the use according to any one of claims 3 to 8; wherein the individual has been determined to have NALFD using a NAFLD fibrosis score.

41. The method according to any one of claims 1 and 4 to 8; the compound according to any one of claims 2 and 4 to 8; or the use according to any one of claims 3 to 8; wherein the individual has been determined to have NALFD by a liver biopsy.

42. The method according to any one of claims 1 and 4 to 8; the compound according to any one of claims 2 and 4 to 8; or the use according to any one of claims 3 to 8; further comprising formulating Compound 1 or a pharmaceutically acceptable salt thereof, into a tablet or capsule.

43. The method according to any one of claims 1 and 4 to 8; the compound according to any one of claims 2 and 4 to 8; or the use according to any one of claims 3 to 8; further comprising formulating Compound 1 or a pharmaceutically acceptable salt thereof, in a tablet or capsule, wherein the tablet or capsule comprises a pharmaceutically acceptable carrier.

44. The method according to any one of claims 1 and 4 to 8; the compound according to any one of claims 2 and 4 to 8; or the use according to any one of claims 3 to 8; wherein the administration of the Compound 1 or a pharmaceutically acceptable salt thereof reduces at least ALT, AST, liver fat, or fatty liver index.
